(19) United States
(12) Patent Application Publication
(10) Pub. No.: US 2024/0226428 A1
(43) Pub. Date: Jul. 11, 2024

(54) WIRELESS PHASE TRANSITION IMPLANTABLE PUMP

(71) Applicant: Cochlear Limited, Macquarie University, NSW (AU)

(72) Inventor: Wolfram Frederik DUECK, Hannover (DE)

(21) Appl. No.: 18/572,080

(22) PCT Filed: Jun. 21, 2022
(86) PCT No.: PCT/IB2022/055763

§ 371 Date: Dec. 19, 2023

Related U.S. Application Data
(60) Provisional application No. 63/220,713, filed on Jul. 12, 2021.

(51) Int. Cl. A61M 5/142

(52) U.S. Cl. CPC A61M 5/14276; A61M 5/14228; A61M 2205/0272; A61M 2205/3334

(57) ABSTRACT

An apparatus includes a housing configured to be implanted on or within a recipient, a cannula at least partially within the housing, and a plurality of flow controllers on or within the housing and in mechanical communication with corresponding portions of the cannula. The plurality of flow controllers is configured to control flow of a material through the portions of the cannula. Each flow controller of the plurality of flow controllers includes a phase-change material configured to, in response to heat, change from a first phase to a second phase and at least one heat source in thermal communication with the phase-change material. The at least one heat source is configured to receive energy from a device external to the recipient and to transmit heat to the phase-change material.

### BACKGROUND

### Field

The present application relates generally to implantable devices configured to provide a liquid treatment substance internally to the recipient, and more specifically, thermally-driven devices for controlling a flow of the liquid treatment substance.

### Description of the Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect disclosed herein, an apparatus is provided which comprises a housing configured to be implanted on or within a recipient, a cannula at least partially within the housing, and a plurality of flow controllers on or within the housing and in mechanical communication with corresponding portions of the cannula. The plurality of flow controllers is configured to control flow of a material through the portions of the cannula. Each flow controller of the plurality of flow controllers comprises a phase-change material configured to, in response to heat, change from a first phase to a second phase, and at least one heat source in thermal communication with the phase-change material. The at least one heat source is configured to receive energy from a device external to the recipient and to transmit heat to the phase-change material.

In another aspect disclosed herein, a method is provided which comprises sequentially transmitting energy from outside a recipient's body to selected flow control elements of a peristaltic flow control system at least partially implanted on or within the recipient's body. The method further comprises sequentially ceasing said transmitting energy to the selected flow control elements. The method further comprises, in response to said transmitting energy and ceasing said transmitting energy, adjusting flow of a material to or from the recipient's body.

In still another aspect disclosed herein, an apparatus is provided which comprises a peristaltic pump configured to be implanted on or within a recipient. The peristaltic pump comprises a cannula configured to repeatedly undergo compression and to be released from compression and a plurality of thermally-activatable pistons in mechanical communication with corresponding portions of the cannula. The plurality of thermally-activatable pistons is configured to receive energy from outside the recipient's body and, in response to the received energy, change shape and/or dimensions so as to compress and/or to release from compression the corresponding portions of the cannula.

In still another aspect disclosed herein, an apparatus is provided which comprises a material configured to be thermally activated and a thermal conduit and/or source implantable on or within a recipient's body. The thermal conduit and/or source comprises a first portion configured to receive energy from a transducer external to the recipient's body and a second portion in thermal communication with the first portion and in thermal communication with the material.

### BRIEF DESCRIPTION OF THE DRAWINGS

Implementations are described herein in conjunction with the accompanying drawings, in which:

FIG. 1 is a perspective view of an example cochlear implant auditory prosthesis implanted in a recipient in accordance with certain implementations described herein;

FIG. 2 schematically illustrates an example apparatus in accordance with certain implementations described herein;

FIGS. 3A-3C schematically illustrate another example apparatus and an example external device in accordance with certain implementations described herein;

FIGS. 4A and 4B schematically illustrate cross-sectional views of an example flow controller in accordance with certain implementations described herein and FIG. 4C schematically illustrates a perspective view of the example flow controller 230 of FIGS. 4A and 4B;

FIG. 5 schematically illustrates an example heat source configured to receive the heat energy from a corresponding transducer in accordance with certain implementations described herein;

FIGS. 6A and 6B schematically illustrate another example apparatus and example device in accordance with certain implementations described herein; and

FIG. 7 is a flow diagram of an example method in accordance with certain implementations described herein.

### DETAILED DESCRIPTION

Certain implementations described herein provide a controllable and implantable pump comprising passive components. The implantable passive delivery system utilizes a plurality of peristaltic pistons comprising a thermally-activatable (e.g., phase-change; phase transition) material, the pistons activated by energy received from an external device worn by the recipient above the implantable pump. The energy from the external device can be in various forms: heat, electrical induction, electromagnetic wave absorption, and/or mechanical friction, and the pistons of the implantable peristaltic pump can comprise an element configured to use the received energy to controllably change the phase of the thermally-activatable material. By having the pump control system outside the recipient's body, certain implementations can avoid implanting non-biocompatible materials of an implantable pump control system into the recipient, thereby avoiding use of hermetic cavities or other schemes to protect the recipient from the non-biocompatible materials. Certain implementations described herein provide more control over the drug release rate than is provided by other passive delivery systems which rely on diffusion or osmotic pressure. By avoiding using electrical components in the implanted pump, certain implementations provide an inexpensive and reliable solution to deliver medicaments locally in a controlled manner over long periods of time.

The teachings detailed herein are applicable, in at least some implementations, to any type of implantable medical device comprising a first portion implanted on or within the recipient's body and configured to provide a medicament to a portion of the recipient's body. The implantable medical device of certain implementations described herein comprises a second portion (e.g., external to a recipient) configured to wirelessly transmit power and/or data to the first portion. Implementations can include any type of medical device that can utilize the teachings detailed herein and/or variations thereof. For example, the medical device can comprise an implantable stimulation system such as auditory prostheses utilizing an implantable actuator assembly including but not limited to: electro-acoustic electrical/acoustic systems, cochlear implant devices, implantable hearing aid devices, middle ear implant devices, bone conduction devices (e.g., active bone conduction devices; passive bone conduction devices, percutaneous bone conduction devices; transcutaneous bone conduction devices), Direct Acoustic Cochlear Implant (DACI), middle ear transducer (MET), electro-acoustic implant devices, other types of auditory prosthesis devices, and/or combinations or variations thereof, or any other suitable hearing prosthesis system with or without one or more external components. For another example, the medical device can comprise an implantable long-term drug delivery device for the vestibular system and inner ear configured to be used to treat a variety of disorders, including but not limited to, chronic or recurring middle ear infections or otitis media (e.g., by administering corticosteroids either regularly in a preventative approach or whenever symptoms arise), progressive age-related hearing loss, noise-induced hearing loss, vertigo, tinnitus, and Meniere's disease. In certain implementations, the teachings detailed herein and/or variations thereof can be utilized in other types of prostheses other than auditory prostheses or as a stand-alone device, in configurations which benefit from long-term reliability (e.g., treatment of chronic conditions throughout the body, such as pain, epilepsy, Parkinson's disease, or other neural disorders) by avoiding implanting non-biocompatible electronic components into the recipient's body.

Merely for ease of description, apparatus and methods disclosed herein are primarily described with reference to an illustrative medical device, namely a cochlear implant auditory prosthesis configured to provide medicament delivery to the middle or inner ear regions. However, the teachings detailed herein and/or variations thereof may also be used with a variety of other medical devices and/or other contexts to provide a wide range of therapeutic benefits to recipients, patients, or other users. In some implementations, the teachings detailed herein and/or variations thereof can be utilized in other types of implantable medical devices beyond auditory prostheses. For example, apparatus and methods disclosed herein and/or variations thereof may also be used with one or more of the following: vestibular devices (e.g., vestibular implants); visual devices (e.g., bionic eyes); visual prostheses (e.g., retinal implants); sensors; cardiac pacemakers; drug delivery systems; defibrillators; functional electrical stimulation devices; catheters; brain implants; seizure devices (e.g., devices for monitoring and/or treating epileptic events); sleep apnea devices; electroporation; pain relief devices; etc.

The teachings detailed herein and/or variations thereof can be utilized in devices configured to deliver medicaments to various portions of the body, including but not limited to: the central nervous system (e.g., spine; brain), muscles, and/or an organ (e.g., heart, lungs, stomach, liver, kidneys, pancreas, eyes). Devices in accordance with certain implementations described herein can be configured to be used for pain treatment (e.g., local pain; chronic pain), intravenous injection for systemic drug therapy of a chronic malady (e.g., insulin for diabetes), and/or in contexts where automatic drug delivery can be used to ensure strict compliance with prescribed dosage levels. Devices in accordance with certain implementations described herein can be configured to be implanted permanently and/or implanted for only a limited time and then removed.

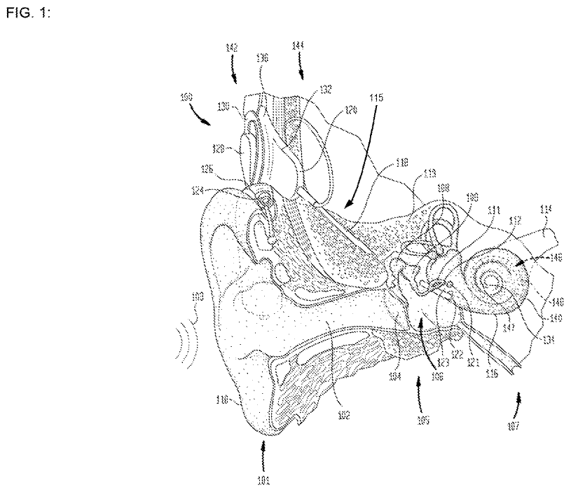

FIG. 1 is a perspective view of an example cochlear implant auditory prosthesis 100 implanted in a recipient in accordance with certain implementations described herein. The example auditory prosthesis 100 is shown in FIG. 1 as comprising an implanted stimulator unit 120 (e.g., an actuator) and an external microphone assembly 124 (e.g., a partially implantable cochlear implant). An example auditory prosthesis 100 (e.g., a totally implantable cochlear implant; a mostly implantable cochlear implant) in accordance with certain implementations described herein can replace the external microphone assembly 124 shown in FIG. 1 with a subcutaneously implantable assembly comprising an acoustic transducer (e.g., microphone).

As shown in FIG. 1, the recipient normally has an outer ear 101, a middle ear 105, and an inner ear 107. In a fully functional ear, the outer ear 101 comprises an auricle 110 and an ear canal 102. An acoustic pressure or sound wave 103 is collected by the auricle 110 and is channeled into and through the ear canal 102. Disposed across the distal end of the ear canal 102 is a tympanic membrane 104 which vibrates in response to the sound wave 103. This vibration is coupled to oval window or fenestra ovalis 112 through three bones of middle ear 105, collectively referred to as the ossicles 106 and comprising the malleus 108, the incus 109, and the stapes 111. The bones 108, 109, and 111 of the middle ear 105 serve to filter and amplify the sound wave 103, causing the oval window 112 to articulate, or vibrate in response to vibration of the tympanic membrane 104. This vibration sets up waves of fluid motion of the perilymph within cochlea 140. Such fluid motion, in turn, activates tiny hair cells (not shown) inside the cochlea 140. Activation of the hair cells causes appropriate nerve impulses to be generated and transferred through the spiral ganglion cells (not shown) and auditory nerve 114 to the brain (also not shown) where they are perceived as sound.

The human skull is formed from a number of different bones that support various anatomical features. Illustrated in FIG. 1 is the temporal bone 115 which is situated at the side and base of the recipient's skull (covered by a portion of the recipient's skin/muscle/fat, collectively referred to herein as tissue). For ease of reference, the temporal bone 115 is referred to herein as having a superior portion and a mastoid portion. The superior portion comprises the section of the temporal bone 115 that extends superior to the auricle 110. That is, the superior portion is the section of the temporal bone 115 that forms the side surface of the skull. The mastoid portion, referred to herein simply as the mastoid bone 119, is positioned inferior to the superior portion. The mastoid bone 119 is the section of the temporal bone 115 that surrounds the middle ear 105.

As shown in FIG. 1, the example auditory prosthesis 100 comprises one or more components which are temporarily or permanently implanted in the recipient. The example auditory prosthesis 100 is shown in FIG. 1 with an external component 142 which is directly or indirectly attached to the recipient's body, and an internal component 144 which is temporarily or permanently implanted in the recipient (e.g., positioned in a recess of the temporal bone 115 adjacent auricle 110 of the recipient). The external component 142 typically comprises one or more input elements/devices for receiving input signals at a sound processing unit 126. The one or more input elements/devices can include one or more sound input elements (e.g., one or more external microphones 124) for detecting sound and/or one or more auxiliary input devices (not shown in FIG. 1)(e.g., audio ports, such as a Direct Audio Input (DAI); data ports, such as a Universal Serial Bus (USB) port; cable ports, etc.). In the example of FIG. 1, the sound processing unit 126 is a Behind-The-Ear sound processing unit configured to be attached to, and worn adjacent to, the recipient's ear. However, in certain other implementations, the sound processing unit 126 has other arrangements, such as by an OTE processing unit (e.g., a component having a generally cylindrical shape and which is configured to be magnetically coupled to the recipient's head), a mini or micro-BTE unit, an in-the-canal unit that is configured to be located in the recipient's ear canal, a body-worn sound processing unit, etc.

The sound processing unit 126 of certain implementations includes a power source (not shown in FIG. 1) (e.g., a battery), a processing module (not shown in FIG. 1) (e.g., comprising one or more digital signal processors (DSPs), one or more microcontroller cores, one or more application-specific integrated circuits (ASICs), firmware, software, etc. arranged to perform signal processing operations), and an external transmitter unit 128. In the illustrative implementations of FIG. 1, the external transmitter unit 128 comprises circuitry that includes at least one external inductive communication coil 130 (e.g., a wire antenna coil comprising multiple turns of electrically insulated single-strand or multi-strand platinum or gold wire). The external transmitter unit 128 also generally comprises a magnet (not shown in FIG. 1) secured directly or indirectly to the at least one external inductive communication coil 130. The at least one external inductive communication coil 130 of the external transmitter unit 128 is part of an inductive radio frequency (RF) communication link with the internal component 144. The sound processing unit 126 processes the signals from the input elements/devices (e.g., microphone 124 that is positioned externally to the recipient's body, in the depicted implementation of FIG. 1, by the recipient's auricle 110). The sound processing unit 126 generates encoded signals, sometimes referred to herein as encoded data signals, which are provided to the external transmitter unit 128 (e.g., via a cable). As will be appreciated, the sound processing unit 126 can utilize digital processing techniques to provide frequency shaping, amplification, compression, and other signal conditioning, including conditioning based on recipient-specific fitting parameters.

The power source of the external component 142 is configured to provide power to the auditory prosthesis 100, where the auditory prosthesis 100 includes a battery (e.g., located in the internal component 144, or disposed in a separate implanted location) that is recharged by the power provided from the external component 142 (e.g., via a transcutaneous energy transfer link). The transcutaneous energy transfer link is used to transfer power and/or data to the internal component 144 of the auditory prosthesis 100. Various types of energy transfer, such as infrared (IR), electromagnetic, capacitive, and inductive transfer, may be used to transfer the power and/or data from the external component 142 to the internal component 144. During operation of the auditory prosthesis 100, the power stored by the rechargeable battery is distributed to the various other implanted components as needed.

The internal component 144 comprises an internal receiver unit 132, a stimulator unit 120, and an elongate electrode assembly 118. In some implementations, the internal receiver unit 132 and the stimulator unit 120 are hermetically sealed within a biocompatible housing, sometimes collectively referred to as a stimulator/receiver unit. The internal receiver unit 132 comprises an internal inductive communication coil 136 (e.g., a wire antenna coil comprising multiple turns of electrically insulated single-strand or multi-strand platinum or gold wire), and generally, a magnet (not shown in FIG. 1) fixed relative to the at least one internal inductive communication coil 136. The at least one internal inductive communication coil 136 receives power and/or data signals from the at least one external inductive communication coil 130 via a transcutaneous energy transfer link (e.g., an inductive RF link). The stimulator unit 120 generates stimulation signals (e.g., electrical stimulation signals; optical stimulation signals) based on the data signals, and the stimulation signals are delivered to the recipient via the elongate electrode assembly 118.

The elongate stimulation assembly 118 has a proximal end connected to the stimulator unit 120, and a distal end implanted in the cochlea 140. The stimulation assembly 118 extends from the stimulator unit 120 to the cochlea 140 through the mastoid bone 119. In some implementations, the stimulation assembly 118 can be implanted at least in the basal region 116, and sometimes further. For example, the stimulation assembly 118 can extend towards apical end of the cochlea 140, referred to as the cochlea apex 134. In certain circumstances, the stimulation assembly 118 can be inserted into the cochlea 140 via a cochleostomy 122. In other circumstances, a cochleostomy can be formed through the round window 121, the oval window 112, the promontory 123, or through an apical turn 147 of the cochlea 140.

The elongate stimulation assembly 118 comprises a longitudinally aligned and distally extending array 146 (e.g., electrode array; contact array) of stimulation elements 148 (e.g., electrical electrodes; electrical contacts; optical emitters; optical contacts). The stimulation elements 148 are longitudinally spaced from one another along a length of the elongate body of the stimulation assembly 118. For example, the stimulation assembly 118 can comprise an array 146 comprising twenty-two (22) stimulation elements 148 that are configured to deliver stimulation to the cochlea 140. Although the stimulation elements 148 of the array 146 can be disposed on the stimulation assembly 118, in most practical applications, the array 146 is integrated into the stimulation assembly 118 (e.g., the stimulation elements 148 of the array 146 are disposed in the stimulation assembly 118). As noted, the stimulator unit 120 generates stimulation signals (e.g., electrical signals; optical signals) which are applied by the stimulation elements 148 to the cochlea 140, thereby stimulating the auditory nerve 114.

While FIG. 1 schematically illustrates an auditory prosthesis 100 utilizing an external component 142 comprising an external microphone 124, an external sound processing unit 126, and an external power source, in certain other implementations, one or more of the microphone 124, sound processing unit 126, and power source are implantable on or within the recipient (e.g., within the internal component 144). For example, the auditory prosthesis 100 can have each of the microphone 124, sound processing unit 126, and power source implantable on or within the recipient (e.g., encapsulated within a biocompatible assembly located subcutaneously), and can be referred to as a totally implantable cochlear implant (“TICI”). For another example, the auditory prosthesis 100 can have most components of the cochlear implant (e.g., excluding the microphone, which can be an in-the-ear-canal microphone) implantable on or within the recipient, and can be referred to as a mostly implantable cochlear implant (“MICI”).

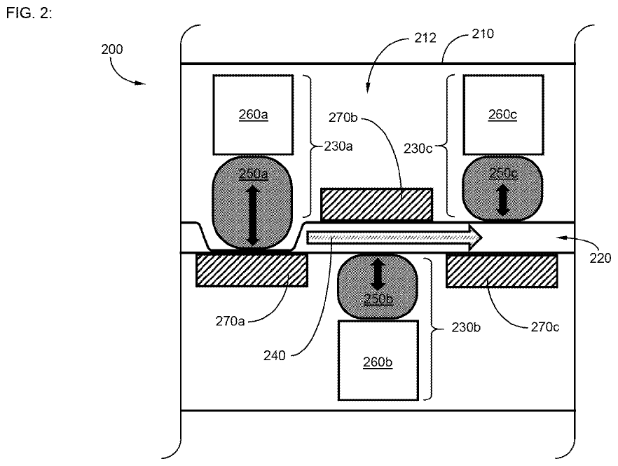

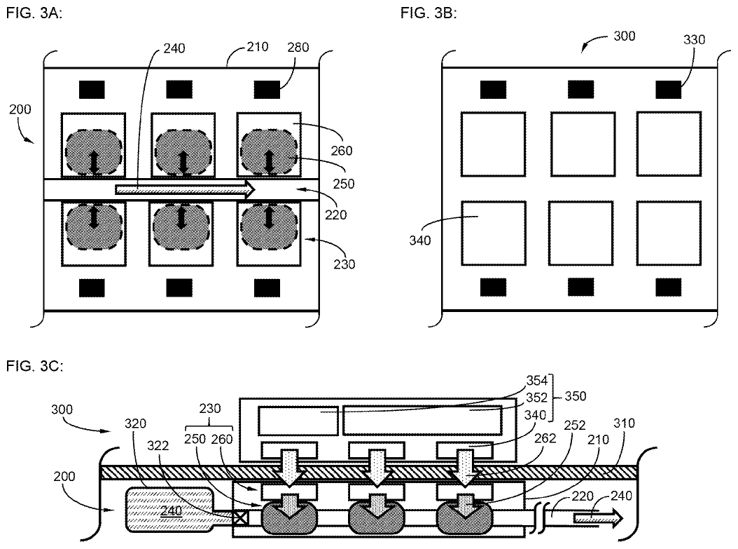

FIG. 2 schematically illustrates an example apparatus 200 in accordance with certain implementations described herein. FIGS. 3A-3C schematically illustrate another example apparatus 200 and an example external device 300 in accordance with certain implementations described herein. The apparatus 200 comprises a housing 210 configured to be implanted on or within a recipient, a cannula 220 at least partially within the housing 210, and a plurality of flow controllers 230 (e.g., 2, 3, 4, 5, 6 or more) on or within the housing 210 and in mechanical communication with corresponding portions of the cannula 220. The plurality of flow controllers 230 is configured to control flow of a material 240 through the portions of the cannula 220. Each flow controller 230 of the plurality of flow controllers 230 comprises a phase-change material 250 and at least one heat source 260 in thermal communication with the phase-change material 250. The phase-change material 250 is configured to, in response to heat 252, change from a first phase to a second phase. The at least one heat source 260 is configured to receive energy 262 from the device 300 external to the recipient and to transmit the heat 252 to the phase-change material 250.

The apparatus 200 of certain implementations is configured to be implanted below tissue 310 (e.g., skin and/or muscle) of the recipient's body and the device 300 is configured to be positioned above the tissue 310 and above the apparatus 200. In certain implementations, the example apparatus 200 and device 300 of FIGS. 3A-3C are components of a system configured to controllably transfer the material 240 to or from a predetermined portion of the recipient's body.

In certain implementations, the flow controllers 230 are configured to be selectively and individually actuated to controllably pump the material 240 from an enclosure 320 (e.g., reservoir) through the cannula 220 to controllably administer the material 240 (e.g., a medicament) internally to the recipient's body. For example, as shown in FIG. 3C, the cannula 220 can be in fluidic communication with the enclosure 320 configured to contain the material 240 (e.g., medicament or treatment substance; in a liquid form, a gel form, and/or comprising nanoparticles or pellets; single constituent material; solution or mixture of two or more constituent materials). In certain implementations, the apparatus 200 comprises the enclosure 320 (e.g., as an integral portion of the apparatus 200), while in certain other implementations, the enclosure 320 is separatable from the other components of the apparatus 200. The material 240 of certain implementations can initially be in a crystalline/solid form that is subsequently dissolved. For example, the enclosure 320 can include two chambers, one that comprises a fluid (e.g., artificial perilymph or saline) and one that comprises the crystalline/solid treatment substance. The fluid can be mixed with the crystalline/solid treatment substance to form a fluid or gel treatment substance that can be subsequently delivered to the recipient.

Alternatively, the flow controllers 230 can be configured to be selectively and individually actuated to controllably draw a sample portion of the material 240 from the recipient's body through the cannula 220 to an enclosure 320 configured to receive the material 240 (e.g., controlled sampling from the recipient), and from which the material 240 can be subsequently extracted from the enclosure 320 for analysis. For example, perilymph liquid can be retrieved and transported (e.g., sucked) from the scala vestibule through the cannula 220 into an enclosure 320 which is accessible through the tissue 310 (e.g., skin). The apparatus 200 can be configured to draw other body fluids (e.g., blood; cerebrospinal fluid) to facilitate diagnostic measures of the drawn body fluids that may be difficult to sample otherwise. For still another example, unwanted fluids can be removed from body spaces which are otherwise difficult to reach (e.g., to treat chronic pericardial effusion). Instead of the enclosure 320, the cannula 220 of the apparatus 200 of certain implementations can be in fluidic communication with a sensor system (e.g., implanted within the recipient's body; separatable from or integral with the apparatus 200) configured to receive and analyze the material 240 drawn from the recipient's body by the apparatus 200.

In certain implementations, the apparatus 200 is integrated with another device implanted in a portion of the recipient's body to provide other benefits or functionalities to the recipient. For example, the apparatus 200 can be integrated with an internal component 144 (e.g., comprising an elongate electrode assembly 118 and a stimulator unit 120) of a cochlear implant auditory prosthesis 100 and can be configured to administer the material 240 to the inner ear 107 of the recipient (e.g., to the cochlea 140 via the round window 121). In certain other implementations, the apparatus 200 can be configured to transfer the material 240 to or from other portions of the recipient's body (e.g., bones; spine; organs; heart; lungs; eyes; liver; brain; stomach; pancreas; kidneys), either separately from or as an integrated part of another implantable device.

As schematically illustrated in FIGS. 3A-3B, the apparatus 200 can comprise one or more magnetic elements 280 (e.g., permanent magnets; ferromagnetic or ferrimagnetic material) and the device 300 can comprise one or more magnetic elements 330 (e.g., permanent magnets; ferromagnetic or ferrimagnetic material). The device 300 can further comprise a plurality of transducers 340 configured to activate and deactivate the plurality of flow controllers 230 of the apparatus 200. The magnetic elements 330 of the device 300 are configured to be aligned with the underlying magnetic elements 280 of the apparatus 200 beneath the tissue 310 and to generate magnetic forces sufficiently strong to hold the device 300 in place above the apparatus 200 with the plurality of transducers 340 positioned over the plurality of flow controllers 230.

In certain implementations, the housing 210 comprises a biocompatible material (e.g., biocompatible metal; titanium; grade 5 titanium; ceramics; glass; polymers) and is configured to be implanted below the tissue 310 (e.g., skin; muscle) of the recipient's body. The housing 210 bounds a region 212 containing the plurality of flow controllers 230. For example, the region 212 can be hermetically sealed by the housing 210 (e.g., sealed from substances in the environment in which the apparatus 200 is implanted).

In certain implementations, the cannula 220 (e.g., fluid conduit; fluid channel; tube; pipe; hose; duct) is configured to transfer material 240 to or from a portion of the recipient's body, with the flow of the material 240 controlled by the plurality of flow control elements 230. For example, as schematically illustrated by FIG. 3C, the cannula 220 can be in fluidic communication with an enclosure 320 and can extend from the enclosure 320 to an outlet port in fluidic communication with the portion of the recipient's body. At least a portion of the cannula 220 has a substantially resilient and flexible wall (e.g., comprising a biocompatible polymer) and is configured to repeatedly be compressed by a force applied by a corresponding flow controller 230 and to return to its uncompressed configuration when the force applied by the corresponding flow controller 230 is removed (e.g., in response to the phase-change material 250 changing between two phases). In certain implementations, the cannula 220 has an outer diameter in a range of 100 microns to 500 microns, an inner diameter in a range of 50 microns to 250 microns, and/or a wall thickness in a range of 25 microns to 150 microns. In its compressed configuration, the cannula 220 reduces (e.g., prevents; blocks) flow of the material 240 through the cannula 220.

In certain implementations, the flow controllers 230 are positioned along the cannula 220 between the enclosure 320 and at least one outlet port of the cannula 220. The flow controllers 230 can be positioned on or within the housing 210 (e.g., wholly outside the housing 210; wholly inside the housing 210; partially outside and partially inside the housing 210; at least partially embedded within a wall of the housing 210). Each flow controller 230 of the plurality of flow controllers 230 is configured to receive energy 262 from the external device 300 and to reversibly change size in response to the energy 262. For example, the flow controllers 230 can expand in response to the energy 262 and can contract in response to absence of the energy 262. In another example, the flow controllers 230 can expand in response to absence of the energy 262 and can contract in response to the energy 262. In certain implementations, the size of a flow controller 230 in the expanded state can be in a range of at least 3%, in a range of 3% to 30%, or in a range of 5% to 15% greater than the size of the flow controller 230 in the non-expanded or contracted state. As schematically illustrated by FIG. 2, each flow controller 230a,b,c can configured to allow flow of the material 240 through a corresponding portion of the cannula 220 when the corresponding phase-change material 250a,b,c is in the first (e.g., non-expanded) phase (see, e.g., flow controllers 230b,c of FIG. 2) and is further configured to not allow flow of the material 240 through the corresponding portion of the cannula 220 when the corresponding phase-change material 250 is in the second (e.g., expanded) phase (see, e.g., flow controller 230a of FIG. 2).

In certain implementations, as schematically illustrated by FIG. 2, the apparatus 200 comprises a plurality of rigid and fixed support portions 270 (e.g., comprising a rigid biocompatible material, such as titanium) positioned such that the cannula 220 extends between each flow controllers 230a,b,c of the plurality of flow controllers 230 and a corresponding support portion 270a,b,c of the plurality of support portions 270. The flow controllers 230 are configured to compress the corresponding portions of the cannula against the corresponding support portions 270a,b,c. In certain implementations, the flow controllers 230a,b,c are on different alternating sides of the cannula 220 (see, e.g., FIG. 2) and the corresponding support portions 270a,b,c are each positioned on an opposite side of the cannula 220 from the corresponding flow controller 230. In certain other implementations, the flow controllers 230 are all on a common side of the cannula 220 and the support portions 270 are on an opposite side of the cannula 220. Two or more of the support portions 270 can be separate from one another, as schematically illustrated by FIG. 2, and/or two or more of the support portions 270 can be integral with one another.

In certain other implementations, as schematically illustrated by FIG. 3A, the cannula 220 extends through pairs of the flow controllers 230, with the two flow controllers 230 of a pair positioned on opposite sides of the cannula 220. The two flow controllers 230 of a pair are configured to be actuated simultaneously with one another, such that the two flow controllers 230 compress the corresponding portion of the cannula 220.

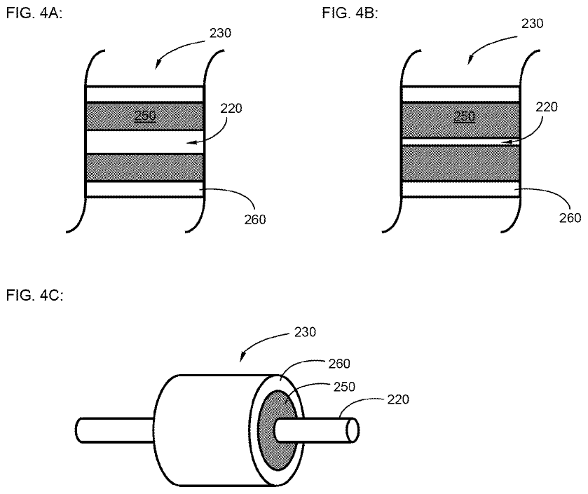

FIGS. 4A and 4B schematically illustrate cross-sectional views of an example flow controller 230 in accordance with certain implementations described herein. FIG. 4C schematically illustrates a perspective view of the example flow controller 230 of FIGS. 4A and 4B. The flow controller 230 of FIGS. 4A-4C comprises a phase-change material 250 that is surrounded by a heat source 260, and the phase-change material 250 surrounds a corresponding portion of the cannula 220. As shown in FIG. 4A, when the flow controller 230 is in a first state, the cannula 220 has a first cross-sectional area. As shown in FIG. 4B, when the flow controller 230 is in a second state, the cannula 220 has a second cross-sectional area smaller than the first cross-sectional area such that flow resistance of the material 240 is greater in the second state than in the first state. In certain implementations, the heat source 260 extends around a perimeter of the phase-change material 250, as schematically illustrated by FIG. 4C, and is configured to constrain the outer perimeter of the phase-change material 250 from expanding radially outward from the cannula 220. In certain other implementations, the heat source 260 also extends over the end portions of the phase-change material 250 such that the heat source 260 fully encloses the phase-change material 250 around the portion of the cannula 220 and is configured to constrain the phase-change material 250 from expanding longitudinally along the cannula 220.

In certain implementations, the phase-change material 250 is within a resiliently flexible container (e.g., comprising a biocompatible polymer) and is configured to respond to the heat 252 from the corresponding heat source 260 by changing from a first (e.g., non-expanded) phase having a size (e.g., length; width; perimeter; area; volume) with a first magnitude to a second (e.g., expanded) phase having a size with a second magnitude different from (e.g., larger than) the first magnitude. The phase-change material 250 of certain implementations is configured to transition between the first phase and the second phase (e.g., between a liquid phase and a gas phase; between a solid phase and a liquid phase) repeatedly, reversibly, and without hysteresis. Examples of phase-change materials 250 compatible with certain implementations described herein include but are not limited to: wax; paraffin; gallium; gallium-iridium alloy; or other material configured to undergo a phase transition in response to a change of temperature. In certain implementations, the phase-change material 250 is configured to transition between the two phases upon a temperature change in a range of 5 to 10 degrees Celsius (e.g., between a temperature at or below 45 degrees Celsius and a temperature at or above 50 degrees Celsius).

In certain implementations, the at least one heat source 260 is configured to receive the energy 262 through the tissue 310 (e.g., transcutaneously; percutaneously) from the external device 300 and to transmit the heat 252 to the phase-change material 250 in response to the energy 262. The at least one heat source 260 can be positioned on or within the housing 210 (e.g., wholly outside the housing 210; wholly inside the housing 210; partially outside and partially inside the housing 210; at least partially embedded within a wall of the housing 210).

In certain implementations, the device 300 comprises circuitry 350 configured to control the apparatus 200 by generating the energy 262 and transmitting the energy 262 through the tissue 310 (e.g., transcutaneously; percutaneously) to the plurality of flow controllers 230. The circuitry 350 of certain implementations comprises the plurality of transducers 340, each transducer 340 configured to generate and transmit the energy 262 to at least one corresponding flow controller 230 of the plurality of flow controllers 230. For example, as schematically illustrated by FIGS. 3A and 3C, the apparatus 200 comprises six flow controllers 230 positioned along the cannula 220 and arranged as three pairs of flow controllers 230 with the cannula 220 extending between the two flow controllers 230 of each pair. The device 300 schematically illustrated by FIGS. 3B-3C comprises six transducers 340 configured to be positioned above the six flow controllers 230 with the tissue 310 therebetween. The six transducers 340 are arranged as three pairs of transducers 340 with the two transducers 340 of each pair configured to simultaneously transmit the energy 262 to both flow controllers 230 of the corresponding pair of flow controllers 230.

FIGS. 3A and 3B show an implementation in which the plurality of heat controllers 230 and the plurality of transducers 340 correspond to one another in a one-to-one configuration (e.g., each heat transducer 340 provides energy 262 to one corresponding flow controller 230 and each flow controller 230 receives energy 262 from one corresponding transducer 340). In certain other implementations, two or more transducers 340 correspond to each flow controller 230 (e.g., multiple transducers 340 provide energy 262 to one corresponding flow controller 230), at least some of the transducers 340 correspond to two or more flow controllers 230 (e.g., at least some of the transducers 340 each provides energy 262 to two or more flow controllers 230), and/or at least some of the flow controllers 230 correspond to two or more transducers 340 (e.g., at least some of the flow controllers 230 receive energy 262 from two or more transducers 340). For example, instead of the three pairs of transducers 340 of FIG. 3B, the device 300 can comprise three transducers 340 each configured to be positioned above a corresponding pair of flow controllers 230 and configured to simultaneously transmit the energy 262 to both flow controllers 230 of the corresponding pair of flow controllers 230.

In certain implementations, the circuitry 350 of the device 300 further comprises a printed-circuit board assembly 352 (e.g., comprising a microprocessor) and at least one power supply 354 (e.g., battery; RF power transfer coil). For example, the printed-circuit board assembly 352 can be in electrical communication with the plurality of transducers 340 and is configured to controllably actuate selected transducers 340 to generate and transmit the energy 262 to selected flow controllers 230 in a sequence configured to controllably pump the material 240 through the cannula 220.

As schematically illustrated in FIG. 2, in certain implementations, each flow controller 230 is configured to selectively respond to the energy 262 from the transducers 340 by expanding and applying a force to a corresponding portion of the cannula 220. The force is sufficient to resiliently compress the portion of the cannula 220 (e.g., against the support portion 270) such that the material 240 in the portion of the cannula 220 is displaced from the portion of the cannula 220 and further flow of the material 240 through the portion of the cannula 220 is reduced (e.g., prevented; blocked). In this way, the flow controllers 230 can be individually and selectively actuated (e.g., activated; expanded) and deactuated (e.g., deactivated; compressed) thereby operating as “pistons” to peristaltically pump the material 240 through the cannula 220.

For example, the flow controllers 230 can initially each be in their non-actuated (e.g., deactivated; non-expanded) state such that none of the corresponding portions of the cannula 220 are compressed. As schematically illustrated in FIG. 2, a first flow controller 230a responds to the energy 262 from a corresponding transducer 340 of the external device 300 by increasing in size (e.g., expanding) in a direction towards the support portion 270a, thereby compressing the corresponding portion of the cannula 220 between the first flow controller 230a and the support portion 270a so as to displace (e.g., force; push) the material 240 from the portion of the cannula 220 (e.g., towards the right in FIG. 2) and to prevent the further flow of the material 240 through the corresponding portion of the cannula 220. When the corresponding transducer 340 ceases providing the energy 262 to the first flow controller 230a (e.g., deactivates the first flow controller 230a), the first flow controller 230a contracts to its smaller size, thereby allowing the corresponding portion of the cannula 220 to elastically return to its non-compressed configuration and permitting flow of the material 240 into and through the corresponding portion of the cannula 220. The other flow controllers (e.g., the second flow controller 230b and the third flow controller 230c shown in FIG. 2) can be similarly configured to selectively respond to the energy 262 from the corresponding transducers 340 so as to displace (e.g., force; push) the material 240 from the corresponding portions of the cannula 220 and to prevent the further flow of the material 240 through the corresponding portions of the cannula 220.

In certain implementations, each flow controller 230 is configured to be individually activated (e.g., expanded) and deactivated (e.g., contracted) independently from the other flow controllers 230. For example, each flow controller 230 can be responsive only to the energy 262 from the corresponding transducer 340 (e.g., each flow controller 230 is unresponsive to energy generated by the other transducers 340). By sequentially activating and deactivating the flow controllers 230a,b,c from left to right in FIG. 2, the material 240 can be peristaltically pumped along the cannula 220 from left to right. For example, starting from a configuration in which none of the three flow controllers 230a,b,c is activated, an example sequence can be: activate the first flow controller 230a to be in a compressed state; activate the second flow controller 230b to be in a compressed state; deactivate the first flow controller 230a to be in a non-compressed state; activate to the third flow controller 230c to be in a compressed state; deactivate the second flow controller 230b to be in a non-compressed state; deactivate the third flow controller 230c to be in a non-compressed state; and repeating the sequence to continue the process of peristaltically pumping the material 240 from the enclosure 320 through the cannula 220 to the recipient. Other schemes for selectively (e.g., sequentially) activating and deactivating the flow controllers 230 are also compatible with certain implementations described herein. In certain implementations, the flow controllers 230 are selectively (e.g., sequentially) actuated and deactivated in accordance with a timing scheme that results in a pumping rate of the material 240 in a range of 10 nanoliters/minute to 500 nanoliters/minute.

Various forms of the energy 262 are compatible with certain implementations described herein. In certain implementations, the energy 262 comprises heat energy and the plurality of transducers 340 comprises a plurality of heating elements configured to generate heat in response to electrical signals received from other portions of the circuitry 350. For example, a heating element can comprise at least one electrical resistor configured to create heat by Joule heating. For another example, a heating element can comprise a thermoelectric element configured to create a temperature differential by the Peltier effect, with a hot side of the thermoelectric element generally facing the tissue 310. In certain implementations, the circuitry 350 is configured to selectively route the driving electrical current among the heating elements to generate and cease generating the heat so as to selectively actuate and deactivate the flow controllers 230.

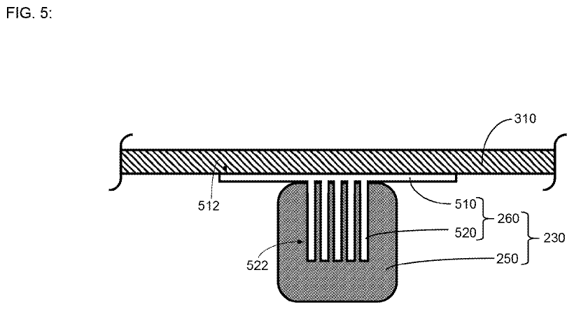

FIG. 5 schematically illustrates an example heat source 260 configured to receive the heat energy 262 from a corresponding transducer 340 in accordance with certain implementations described herein. The heat source 260 comprises a first portion 510 configured to receive heat from the corresponding transducer 340 and a second portion 520 in thermal communication with the first portion 510 and in thermal communication with the phase-change material 250. For example, the first portion 510 can comprise a first surface 512 facing the corresponding transducer 340. The first surface 512 can have a first surface area that is sufficiently large such that the heat density applied by the transducer 340 to the tissue 310 is below a predetermined threshold (e.g., pain threshold; tissue damage threshold) while providing sufficient heat to actuate the flow controller 230. The second portion 520 can have a second surface 522 in contact with the phase-change material 250 and having a second surface area that is sufficiently large such that the heat received by the heat source 260 from the corresponding transducer 340 is transferred with sufficient efficiency to the phase-change material 250 to actuate the flow controller 230. In certain implementations, the first portion 510 and the second portion 520 each comprise a thermally conductive material (e.g., copper; aluminum) and are integral with one another (e.g., forming a unitary element). The heat source 260 of certain implementations is configured to facilitate heat transfer between the transducer 340 and the phase-change material 250. For example, as schematically illustrated by FIG. 5, the first surface 512 can be substantially flat and generally parallel to the tissue 310 between the apparatus 200 and the device 300 (e.g., between the first surface 512 and the transducer 340) and the second surface 522 can comprise a plurality of protrusions (e.g., ribs) and/or is corrugated. In certain implementations, the heat source 260 comprises a thermally insulative coating on portions of the heat source 260 that are not part of the first or second surfaces 512, 522 and are not in thermal communication with the phase-change material 250 (e.g., to reduce or prevent heat loss from the heat source 260 from reaching the phase-change material 250).

In certain implementations, instead of the plurality of transducers 340 being configured to generate and transmit energy 262 to the plurality of flow controllers 230, the plurality of transducers 340 can be configured to individually and selectively cool the plurality of flow controllers 230 (e.g., operating as a heat sink). Instead of heat sources 260, the flow controllers 230 of certain such implementations comprise thermally conductive elements configured to transfer heat from the phase-change material 250 through the tissue 310 to a corresponding transducer 340, thereby cooling the phase-change material 250. For example, the transducer 340 can comprise a thermoelectric element configured to create a temperature differential by the Peltier effect, with a cold side of the thermoelectric element generally facing the tissue 310. The thermally conductive element of certain such implementations can have the structure of the heat source 260 schematically illustrated by FIG. 5, but instead of transferring heat from the transducer 340 to the phase-change material 250, the thermally conductive element transfers heat from the phase-change material 250 to the transducer 340. In certain implementations in which the transducers 340 comprise thermoelectric elements, the circuitry 350 of the device 300 can be configured to selectively drive the transducers 340 to heat and/or cool the phase-change material 250 via the thermally conductive heat source 260 as appropriate to activate and/or deactivate the flow controllers 230.

In certain implementations, the energy 262 comprises electromagnetic energy and the plurality of transducers 340 comprises a plurality of electromagnets configured to generate time-varying magnetic fields in response to alternating electrical signals received from other portions of the circuitry 350. In certain other implementations, the plurality of transducers 340 comprise a plurality of generators (e.g., microwave sources) configured to generate electromagnetic waves in response to electrical signals received from other portions of the circuitry 350. The circuitry 350 can be configured to selectively route the driving electrical signal among the transducers 340 to generate and cease generating the energy 262 so as to selectively actuate and deactivate the flow controllers 230.

In certain implementations, the time-varying magnetic fields and/or the electromagnetic waves are configured to generate electrical eddy currents within the heat source 260 of the corresponding flow controller 230, and the heat source 260 comprises an electrically conductive portion in which the electrical eddy currents flow, thereby creating heat 252 by the Joule effect, with the heat 252 being transferred to the phase-change material 250. For example, the heat source 260 can have the structure schematically illustrated by FIG. 5, with the first portion 510 comprising an electrically conductive material (e.g., copper; aluminum). The shape and/or dimensions of the heat source 260 can be configured to facilitate electrical eddy currents within the heat source 260, which can be different from the shape and/or dimensions of the heat source 260 configured to receive heat from the transducer 340 (e.g., the first surface 512 not configured to facilitate heat transfer through the tissue 310). For another example, the heat source 260 can comprise electrically conductive particles within the phase-change material 250, the particles configured to create heat 252 by eddy current flow within the particles.

In certain other implementations, the plurality of transducers 340 comprises a plurality of antennas 610 configured to generate time-varying magnetic fields and/or electromagnetic waves in response to electrical signals received from other portions of the circuitry 350. The circuitry 350 can be configured to selectively route the driving electrical signal among the antennas 610 to generate and cease generating the energy 262 so as to selectively actuate and deactivate the flow controllers 230. The time-varying magnetic field generated by an antenna 610 can be configured to generate a time-varying magnetic flux, at least a portion of which is received by a heat source 260 of the corresponding flow controller 230.

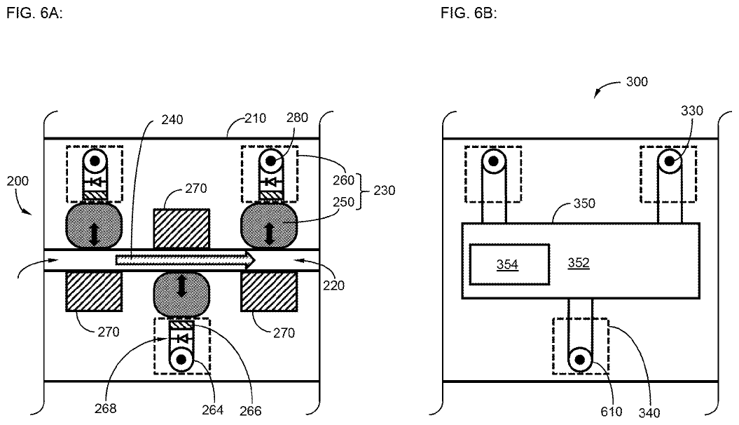

FIGS. 6A and 6B schematically illustrate another example apparatus 200 and example device 300 in accordance with certain implementations described herein. The plurality of transducers 340 of FIG. 6B comprises a plurality of antennas 610 configured to receive alternating electrical signals from the printed-circuit board assembly 352 of the circuitry 350 and to generate the time-varying magnetic fields for actuating and/or deactivating the flow controllers 230 of the apparatus 200. In certain implementations, the antennas 610 comprise at least one electrically conductive coil and can further comprise a ferromagnetic or ferrimagnetic core around which the coil is wound.

As schematically illustrated by FIG. 6A, the heat source 260 can comprise an electrically conductive coil 264 configured to receive at least a portion of the time-varying magnetic flux from the corresponding antenna 610 (e.g., in inductive communication with the corresponding antenna 610) and to generate an alternating electrical current that flows through at least one electrical resistor 266 of the heat source 260, thereby creating heat 252 by the Joule effect, the at least one electrical resistor 266 in thermal communication with the phase-change material 250. In certain implementations, the heat source 260 can further comprise a rectifier 268 (e.g., diode) in parallel with the electrically conductive coil 264 and the at least one electrical resistor 266. While FIG. 6A shows an example implementation in which the electrically conductive coil 264 and the at least one electrical resistor 266 are discrete components, in certain other implementations, the electrically conductive coil 264 can be in thermal communication with (e.g., wrapped around) the phase-change material 250 and can have sufficient electrical resistance to serve as the at least one electrical resistor 266 by generating the heat 252 by the Joule effect and providing the heat 252 to the phase-change material 250.

As schematically illustrated by FIGS. 6A and 6B, the apparatus 200 can comprise a plurality of magnetic elements 280 (e.g., concentric with the electrically conductive coils 264 of the flow controllers 230) and the device 300 can comprise a plurality of magnetic elements 330 (e.g., concentric with the electrically conductive coils of the antennas 610) configured to align the transducers 340 with the underlying flow controllers 230 and to generate magnetic forces sufficiently strong to hold the device 300 in place above the apparatus 200.

In certain implementations, the energy 262 comprises vibrational energy and the plurality of transducers 340 comprises a plurality of actuators configured to generate vibrations in response to electrical signals received from other portions of the circuitry 350. The circuitry 350 can be configured to selectively route the driving electrical signals among the actuators to generate and cease generating the vibrations so as to selectively actuate and deactivate the flow controllers 230. In certain implementations, the heat sources 260 are configured to receive the vibrations that propagate through the tissue 310 and to generate the heat 252 by mechanical friction. For example, the heat source 260 can comprise at least two structures configured to rub against one another due to the received vibrations, with sufficient friction to generate the heat 252 that is transferred to the phase-change material 250. For another example, the phase-change material 250 itself can serve as the heat source 260 by having portions of the phase-change material 250 create the heat 252 by friction.

In certain implementations, the enclosure 320 is implanted within the recipient's body (e.g., within the housing 210; outside the housing 210; separatable from or integral with the apparatus 200). The enclosure 320 can comprise a biocompatible material (e.g., silicone) configured to be positioned below the tissue 312 (e.g., between layers of the recipient's tissue or adjacent to a subcutaneous outer surface of the recipient's skull; positioned in a surgically created pocket at the outer surface adjacent to a superior portion of the temporal bone). In certain implementations, the enclosure 320 is in fluidic communication with the cannula 220 via a selectively actuated valve 322 (e.g., within the housing 210; outside the housing 210) that is configured to be opened or closed in response to signals from the device 300. The valve 322 can be configured to only allow flow of the material 240 from the enclosure 320 to the cannula 220 when the device 300 is properly positioned on the recipient's body relative to the apparatus 200. In certain implementations, at least one flow controller 230 is configured to be used as the selectively actuated valve 322 to controllably allow flow of the material 240 into the cannula 220 from the enclosure 320 or to controllably prevent flow of the material 240 into the cannula 220. In certain other implementations, the selectively actuated valve 322 comprises a magnetic element that is configured to open the valve 322 when actuated by a corresponding magnetic element of the device 300. The magnetic element of the device 300 can be a permanent magnet configured to only actuate (e.g., open) the valve 322 when the device 300 is in proper operating position relative to the apparatus 200, or the magnetic element of the device 300 can be an electromagnet configured to be controlled by the circuitry 350 of the device 300 to selectively actuate (e.g., open) the valve 322 to controllably administer the material 240 from the enclosure 320 to the recipient's body.

In certain implementations, the enclosure 320 is, prior to or after implantation, at least partially filled and/or refilled with the material 240 for delivery to the recipient. For example, the enclosure 320 can comprise a septum (e.g., needle port; comprising silicone or an elastomer) configured to be punctured by a needle from outside the recipient's body and extending through the tissue 312 into the enclosure 320. In certain other implementations, the enclosure 320 is configured to be explanted and replaced with another enclosure that is, prior to or after implantation, at least partially filled with a treatment substance.

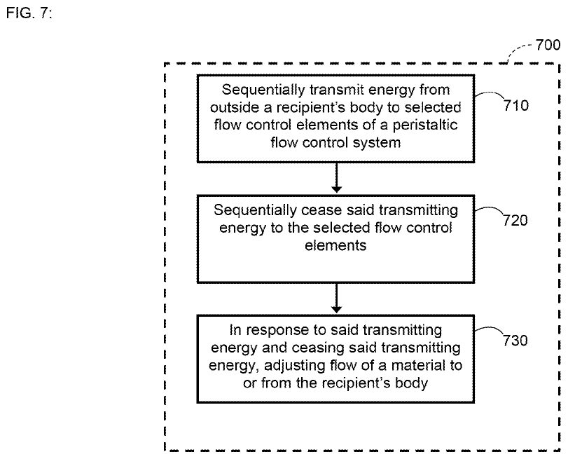

FIG. 7 is a flow diagram of an example method 700 in accordance with certain implementations described herein. While the method 700 is described by reference to the example apparatus 200 and example device 300 of FIGS. 2, 3A-3C, 4A-4C, 5, and 6A-6B, other apparatus, elements, and structures are also compatible with the method 700. In an operational block 710, the method 700 comprises sequentially transmitting energy 262 from outside a recipient's body to selected flow control elements (e.g., flow controllers 230) of a peristaltic flow control system (e.g., apparatus 200) at least partially implanted on or within the recipient's body. In an operational block 720, the method 700 further comprises sequentially ceasing said transmitting energy 262 to the selected flow control elements. In an operational block 730, the method 700 further comprises, in response to said transmitting energy and ceasing said transmitting energy, adjusting flow of a material 240 to or from the recipient's body.

In certain implementations, the method 700 further comprises sequentially applying heat 252 to a thermally activated material (e.g., phase-change material 250) of the selected flow control elements and sequentially ceasing applying the heat 252 to the thermally activated material of the selected flow control elements. For example, the thermally activated material can be configured to change shape and/or size in response to the heat 252. Each flow control element can in mechanical communication with a corresponding portion of a cannula 220 and the thermally activated material can be responsive to the heat 252 by changing from a first phase to a second phase. The corresponding portion of the cannula 220 is compressed when the thermally activated material is in one of the first and second phases and the corresponding portion of the cannula is not compressed when the thermally activated material is in the other of the first and second phases. For example, the thermally activated material in the first phase can have a size with a first magnitude and the thermally activated material in the second phase can have a size with a second magnitude different from the first magnitude.

In certain implementations, the energy 262 transmitted from outside the recipient's body comprises the heat 252 applied to the thermally activated material. In certain other implementations, the energy 262 transmitted from outside the recipient's body comprises electromagnetic and/or vibratory energy 262 and the flow control elements are configured to respond to the electromagnetic and/or vibratory energy 262 by generating the heat 252.

Although commonly used terms are used to describe the systems and methods of certain implementations for ease of understanding, these terms are used herein to have their broadest reasonable interpretations. Although various aspects of the disclosure are described with regard to illustrative examples and implementations, the disclosed examples and implementations should not be construed as limiting. Conditional language, such as, among others, “can,” “could,” “might,” or “may,” unless specifically stated otherwise, or otherwise understood within the context as used, is generally intended to convey that certain implementations include, while other implementations do not include, certain features, elements and/or steps. Thus, such conditional language is not generally intended to imply that features, elements and/or steps are in any way required for one or more implementations or that one or more implementations necessarily include logic for deciding, with or without user input or prompting, whether these features, elements and/or steps are included or are to be performed in any particular implementation. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

It is to be appreciated that the implementations disclosed herein are not mutually exclusive and may be combined with one another in various arrangements. In addition, although the disclosed methods and apparatuses have largely been described in the context of conventional cochlear implants, various implementations described herein can be incorporated in a variety of other suitable devices, methods, and contexts.

Language of degree, as used herein, such as the terms “approximately,” “about,” “generally,” and “substantially,” represent a value, amount, or characteristic close to the stated value, amount, or characteristic that still performs a desired function or achieves a desired result. For example, the terms “approximately,” “about,” “generally,” and “substantially” may refer to an amount that is within +10% of, within +5% of, within +2% of, within +1% of, or within +0.1% of the stated amount. As another example, the terms “generally parallel” and “substantially parallel” refer to a value, amount, or characteristic that departs from exactly parallel by +10 degrees, by +5 degrees, by +2 degrees, by +1 degree, or by +0.1 degree, and the terms “generally perpendicular” and “substantially perpendicular” refer to a value, amount, or characteristic that departs from exactly perpendicular by +10 degrees, by +5 degrees, by +2 degrees, by +1 degree, or by +0.1 degree. The ranges disclosed herein also encompass any and all overlap, sub-ranges, and combinations thereof. Language such as “up to,” “at least,” “greater than,” less than,” “between,” and the like includes the number recited. As used herein, the meaning of “a,” “an,” and “said” includes plural reference unless the context clearly dictates otherwise. Also, as used in the description herein, the meaning of “in” includes “into” and “on,” unless the context clearly dictates otherwise.

While the methods and systems are discussed herein in terms of elements labeled by ordinal adjectives (e.g., first, second, etc.), the ordinal adjective are used merely as labels to distinguish one element from another (e.g., one signal from another or one circuit from one another), and the ordinal adjective is not used to denote an order of these elements or of their use.

The invention described and claimed herein is not to be limited in scope by the specific example implementations herein disclosed, since these implementations are intended as illustrations, and not limitations, of several aspects of the invention. Any equivalent implementations are intended to be within the scope of this invention. Indeed, various modifications of the invention in form and detail, in addition to those shown and described herein, will become apparent to those skilled in the art from the foregoing description. Such modifications are also intended to fall within the scope of the claims. The breadth and scope of the invention should not be limited by any of the example implementations disclosed herein, but should be defined only in accordance with the claims and their equivalents.

1. An apparatus comprising:
a housing configured to be implanted on or within a recipient;
a cannula at least partially within the housing; and
a plurality of flow controllers on or within the housing and in mechanical communication with corresponding portions of the cannula, the plurality of flow controllers configured to control flow of a material through the portions of the cannula, each flow controller of the plurality of flow controllers comprising:
a phase-change material configured to, in response to heat, change from a first phase to a second phase; and
at least one heat source in thermal communication with the phase-change material, the at least one heat source configured to receive energy from a device external to the recipient and to transmit heat to the phase-change material.

2. The apparatus of claim 1, wherein the material comprises at least one medicament and the flow of the material through the cannula comprises controlled administration of the at least one medicament internally to the recipient.

3. The apparatus of claim 2, further comprising an enclosure in fluidic communication with the cannula, the enclosure configured to contain the material, wherein the cannula is configured to flow the material from the enclosure to a location on or within the recipient.

4. The apparatus of claim 1, wherein the material comprises a body liquid from the recipient and the flow of the material through the cannula comprises controlled sampling of the body liquid internally from the recipient.

5. The apparatus of claim 1, wherein the at least one heat source comprises at least one thermal conduit in thermal communication with the phase-change material, the at least one thermal conduit configured to receive heat from the device and to transmit the heat to the phase-change material.

6. The apparatus of claim 1, wherein the at least one heat source comprises at least one electrically conductive element in thermal communication with the phase-change material, the at least one electrically conductive element configured to respond to electromagnetic energy from the device by flowing eddy currents through the at least one electrically conductive element to generate and transmit the heat to the phase-change material.

7. The apparatus of claim 1, wherein the at least one heat source comprises at least one actuator in thermal communication with the phase-change material, the at least one actuator configured to respond to electromagnetic and/or vibratory energy from the device to generate and transmit the heat to the phase-change material.

8. The apparatus of claim 1, wherein the at least one heat source comprises at least one electrically conductive coil and at least one electrical resistor in electrical communication with the at least one electrically conductive coil and in thermal communication with the phase-change material, the at least one electrically conductive coil configured to respond to electromagnetic energy from the device by flowing electrical current through the at least one electrical resistor to generate and transmit the heat to the phase-change material.

9. The apparatus of claim 1, further comprising a plurality of first ferromagnetic elements configured to generate attracting magnetic forces with a corresponding plurality of second ferromagnetic elements of the device external to the recipient such that the device is held by the magnetic forces onto the recipient in a predetermined orientation relative to the plurality of flow controllers.

10. The apparatus of claim 1, wherein the flow controllers are configured to be selectively actuated to peristaltically pump the material through the at least one cannula.

11. A method comprising:
sequentially transmitting energy from outside a recipient's body to selected flow control elements of a peristaltic flow control system at least partially implanted on or within the recipient's body;
sequentially ceasing said transmitting energy to the selected flow control elements; and
in response to said transmitting energy and ceasing said transmitting energy, adjusting flow of a material to or from the recipient's body.

12. The method of claim 11, further comprising sequentially applying heat to the selected flow control elements and sequentially ceasing applying the heat to the selected flow control elements.

13. The method of claim 12, wherein the energy comprises the heat.

14. The method of claim 12, wherein the energy comprises electromagnetic and/or vibratory energy and the flow control elements are configured to respond to the electromagnetic and/or vibratory energy by generating the heat.

15. The method of claim 11, wherein each flow control element comprises a thermally activated material configured to change shape and/or size in response to heat.

16. The method of claim 15, wherein each flow control element is in mechanical communication with a corresponding portion of a cannula and the thermally activated material is responsive to the heat by changing from a first phase to a second phase, the thermally activated material in one of the first and second phases compresses the corresponding portion of the cannula and the thermally activated material in the other of the first and second phases does not compress the corresponding portion of the cannula.

17. The method of claim 16, wherein the thermally activated material in the first phase has a size with a first magnitude and the thermally activated material in the second phase has a size with a second magnitude different from the first magnitude.

18. An apparatus comprising:
a peristaltic pump configured to be implanted on or within a recipient, the peristaltic pump comprising:
a cannula configured to repeatedly undergo compression and to be released from compression; and
a plurality of thermally-activatable pistons in mechanical communication with corresponding portions of the cannula, the plurality of thermally-activatable pistons configured to receive energy from outside the recipient's body and, in response to the received energy, change shape and/or dimensions so as to compress and/or to release from compression the corresponding portions of the cannula.

19. The apparatus of claim 18, wherein the apparatus further comprises an enclosure in fluidic communication with the cannula, the enclosure configured to provide a fluid material to the cannula and/or to receive a fluid material from the cannula.

20. The apparatus of claim 19, wherein the enclosure is configured to provide a fluid medicament to the cannula and the cannula is configured to provide the fluid medicament to the recipient's body.

21. The apparatus of claim 19, wherein the cannula is configured to receive a body fluid from the recipient's body and the enclosure is configured to receive the body fluid from the cannula.

22. The apparatus of claim 18, wherein the received energy is transmitted transcutaneously from outside the recipient's body to the plurality of thermally-activatable pistons.

23. The apparatus of claim 18, wherein each thermally-activatable piston of the plurality of thermally-activatable pistons comprises a phase-change material configured to, in response to heat, change from a first phase having a first shape to a second phase having a second shape different from the first shape.

24. The apparatus of claim 23, wherein the received energy comprises heat energy and each thermally-activatable piston of the plurality of thermally-activatable pistons comprises a heat conduit configured to transmit at least a portion of the received heat energy to the phase-change material.

25. The apparatus of claim 18, wherein the received energy comprises electromagnetic and/or vibratory energy and each thermally-activatable piston of the plurality of thermally-activatable pistons comprises a heat generator configured to generate heat in response to the received energy and to transmit the generated heat to the phase-change material.

26. An apparatus comprising:
a material configured to be thermally activated; and
a thermal conduit and/or source implantable on or within a recipient's body, the thermal conduit and/or source comprising:
a first portion configured to receive energy from a transducer external to the recipient's body; and
a second portion in thermal communication with the first portion and in thermal communication with the material.

27. The apparatus of claim 26, wherein the energy comprises heat energy and the thermal conduit and/or source comprises a thermally conductive material configured to transmit at least a portion of the heat energy to the material.

28. The apparatus of claim 26, wherein the energy comprises electromagnetic and/or vibratory energy and the thermal conduit and/or source is configured to convert at least a portion of the electromagnetic and/or vibratory energy into heat energy and to transmit at least a portion of the heat energy to the material.

29. The apparatus of claim 26, wherein the first portion comprises a first surface facing the transducer.

30. The apparatus of claim 29, wherein the first surface is substantially flat and generally parallel to tissue between the first surface and the transducer.

31. The apparatus of claim 26, wherein the second portion has a second surface in contact with the material.

32. The apparatus of claim 31, wherein the second surface comprises a plurality of protrusions and/or is corrugated.

33. The apparatus of claim 26, wherein the material comprises a phase-change material configured to, in response to heat, change from a first phase having a first shape to a second phase having a second shape different from the first shape.

34. The apparatus of claim 26, wherein the first portion and the second portion each comprise a thermally conductive material and are integral with one another.

35. The apparatus of claim 26, wherein the thermal conduit and/or source comprises a thermally insulative coating on portions of the thermal conduit and/or source that are not configured to receive the energy from the transducer and that are not in thermal communication with the material.
